(19) United States
(12) Patent Application Publication
(10) Pub. No.: US 2006/0229593 A1
(43) Pub. Date: Oct. 12, 2006

(54) Surgical Illumination insert

(75) Inventors: Alex Vayser, Mission Viejo, CA (US); Kenneth Trauner, San Francisco, CA (US)

Correspondence Address: Crockett & Crockett, Suite 400, 24012 Calle De La Plata, Laguna Hills, CA, 92653, US

(73) Assignee: Spotlight Surgical, Inc.

(21) Appl. No.: 11/356,894

(22) Filed: Feb. 17, 2006

Related U.S. Application Data
(60) Provisional application No. 60/654,356, filed on Feb. 18, 2005.

(51) Int. Cl. A61B 18/18

(52) U.S. Cl. 606/4

(57) ABSTRACT

A surgical illumination insert according to the present disclosure may include one or more illumination elements mounted on an insert body that may also include control electronics. The surgical illumination insert affords controllable direct illumination of a deep cavity surgical site and may be moved independent of any surgeon or surgical instrument. The insert body provides heat conductivity to remove heat generated by the illumination elements. The insert body is curved for insertion through surgical retractors and to afford optimal heat transfer to the retractor. Energy for the illumination elements may be provided by batteries through a cable or wireless link.

### RELATED APPLICATIONS

This application claims priority of copending U.S. provisional patent application Ser. No. 60/654,356 filed Feb. 18, 2005.

### FIELD OF THE INVENTIONS

The inventions described below relate the field of surgical instruments, and more specifically to illumination inserts for use in surgical retractors.

### BACKGROUND OF THE INVENTIONS

Existing technology for illumination during surgical/medical procedures is currently limited to over-head illumination. This illumination comes from either overhead lighting or head mounted fiber optic systems. Traditional overhead lighting systems face numerous limitations. Direct exposure of the surgical field from the overhead source is required. Changes in patient or surgeon positioning may interfere with the field illumination. Frequent adjustment of the light source represents an inconvenience for the surgeon and disrupts the surgical flow. Overhead lighting is frequently inadequate for surgery in deeper cavities where more intense focused illumination may be required. In addition, where multiple surgeons are participating, the alignment of the surgeon's head frequently interferes with the remote illumination and prevents light from reaching the surgical field.

Head mounted fiber optic systems are used frequently for more limited surgical exposures. However, head mounted fiber optic systems have limitations. For example, the light cord attached to the headset tethers the surgeon, limiting mobility in the operating room. Fiber optic devices are often associated with head and neck fatigue from frequent or prolonged use. Head mounted fiber optic systems may also require the surgeon to maintain a steady head neck position to provide a constant and steady illumination of the field. Also, the use of remote light sources and fiber bundles introduces tremendous inefficiencies into the system. An approximate 10% loss per foot of cable a 300 Watt light source and a 10 ft cable will only provide just a few watts of illumination. The introduction and popularity of minimally invasive surgical techniques, has raised the demand for the delivery of high intensity light through minimal surgical incisions into deep cavity surgical fields.

Some light delivery devices have been developed for delivery of light from a remote, high intensity light sources to the surgical field. Conventional devices consist of bundles of optical fibers directly attached to surgical retractors to illuminate the surgical field. These light delivery devices are connected via fiber optic cable to a high intensity light source. While these devices provide a technique for directly illuminating the surgical field, they are cumbersome. Having these conventional devices directly tethered to the retractors, they provide limited illumination. The fiber bundles are inconvenient, interfere with access, destabilize the retractor positioning, and they provide inefficient illumination.

What is needed is a source of surgical illumination for deep cavity surgery that may be directed independent of the surgeon and the surgical instruments.

### SUMMARY

Recently, advances in light emitting diode (LED) technology have generated the potential to provide high intensity illumination from a small device. A surgical illumination insert according to the present disclosure may include a light source such as an LED, control electronics, energy source, and a heat sink. This illumination insert may be compatible with standard surgical retractors permitting the insert to be placed through the retractor and provide direct illumination to a deep cavity surgical site. Illumination provided by a device independent of the surgeon and the surgical tools permits the illumination to be directed as needed with minimal interference and without limiting access to the surgical site.

A surgical illumination insert according to the present disclosure may include one or more illumination elements mounted on an insert body that may also include control electronics. The surgical illumination insert affords controllable direct illumination of a deep cavity surgical site and may be moved independent of any surgeon or surgical instrument. The insert body provides heat conductivity to remove heat generated by the illumination elements. The insert body is curved for insertion through surgical retractors and to afford optimal heat transfer to the retractor. Energy for the illumination elements may be provided by batteries through a cable or wireless link.

A surgical illumination apparatus according to the present disclosure may include a heat sink configured to be compatible with a surgical retractor, one or more light elements secured to the heat sink, an energy source to illuminate the light elements and a control element controlling the application of energy to the one or more light elements.

A surgical retractor insert may include an insert body compatible with the surgical retractor, one or more light elements secured to the insert body, an energy source to illuminate the light elements, and a control element controlling the application of energy to the one or more light elements.

### BRIEF DESCRIPTION OF THE DRAWINGS

FIG. 1 is a high level block diagram of a surgical illumination insert according to the present disclosure.

FIG. 2 is a front view of a surgical illumination insert according to the present disclosure.

FIG. 3 is an end view of the surgical illumination insert of FIG. 2.

### DETAILED DESCRIPTION OF THE INVENTIONS

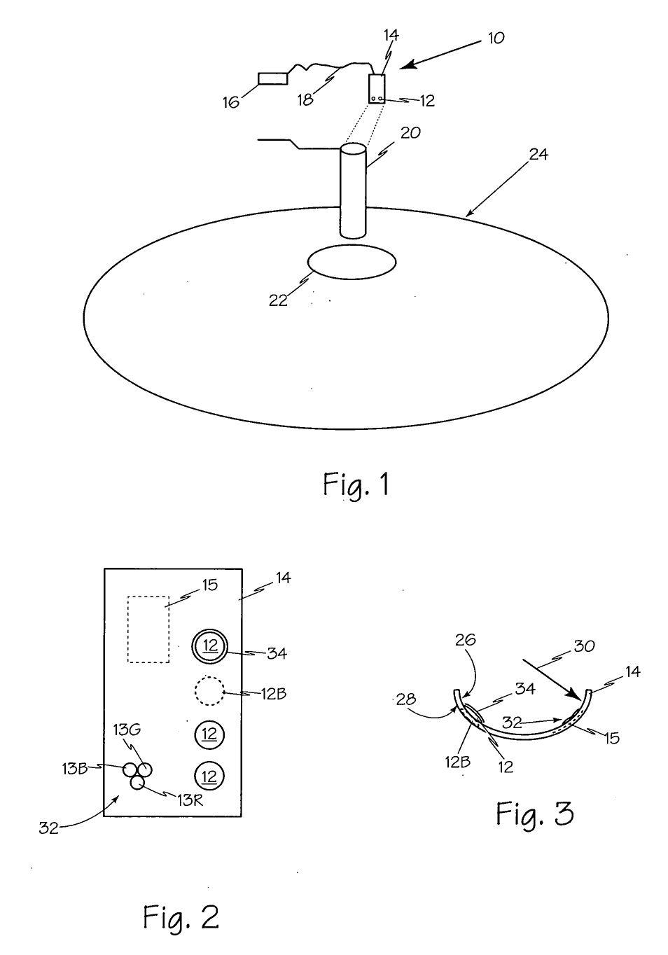

Referring now to FIG. 1, surgical illumination insert 10 includes one or more illumination or light elements such as LED 12 mounted on, or molded or inserted within insert body 14. Power source 16 may provide energy to surgical illumination insert 10 through any suitable cable 18 or through a wireless link.

In use, surgical illumination insert 10 may be inserted through a surgical retractor such as retractor 20 to provide illumination to surgical site 22 within body 24. Insert 10 may be moved within retractor 20 to provide illumination as needed and afford the surgeon an optimal view of surgical site 22 and adjacent tissue.

Referring now to FIGS. 2 and 3, insert body 14 provides a mounting surface for one or more light elements such as LED 12. Alternatively, one or more light elements such as LED 12 may be molded or located within insert body 14. Insert body 14 also operates as a heat sink to conduct heat generated by the illumination elements from the surgical site. Insert body 14 may adopt any suitable geometry, such as a radius of curvature 30 corresponding to one or more retractors for which it is suited. Use of an appropriate radius of curvature 30 may permit insert body 14 to transfer heat drawn from LEDs 12 to a surgical retractor such as retractor 20.

Light elements may be any suitable incandescent or solid state devices such as LEDs 12 and 12B that may be secured to, or visible through either or both sides 26 and 28, and they may adopt any suitable orientation relative to each other. Light elements may have any suitable configuration and or color, such as a white surface mount LED. Other colors or combinations of colors for light elements may permit selectable frequency of illumination for a surgical site to enable illumination or therapy. For example, array 32 may include a red LED 13R, a green LED 13G, and a blue LED 13B or any other suitable combination. The array elements such as LEDs 13R, 13G and 13B may be collocated in any suitable arrangement to enable any combination of the microchips or other light element to be illuminated by control electronics for illumination or therapeutic benefit.

Alternatively, light elements 12 may be organic or polymer LEDs, OLED, PLED respectively, that may have another diffuser film applied on top of the OLED film to direct the light in a desired direction and/or shape the light in a desired shape, such as circular, square, rectangle, triangle, etc.

A surgical illumination system according to the present disclosure may also include any suitable layers, lenses, films or other to modify or control the light from light elements 12. For example, film 34 may be overlaid on one or more light elements 12 to provide polarized light. Any suitable combination of films, layers and lenses may be used.

Control electronics 15 may be contained within insert body 14 or included with power source 16 in a common housing.

Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A surgical retractor insert comprising:
an insert body compatible with the surgical retractor;
one or more light elements secured to the insert body;
an energy source to illuminate the light elements;
a control element controlling the application of energy to the one or more light elements.

2. The surgical retractor insert of claim 1 wherein the one or more light elements are solid state devices.

3. The surgical retractor insert of claim 1 wherein the energy source conveys energy to the one or more light elements using a wireless link.

4. The surgical retractor insert of claim 1 wherein the one or more light elements further comprise:
one or more arrays of light elements.

5. A surgical illumination apparatus comprising:
a heat sink configured to be compatible with a surgical retractor;
one or more light elements secured to the heat sink;
an energy source to illuminate the light elements;
a control element controlling the application of energy to the one or more light elements.

6. The apparatus of claim 5 wherein the one or more light elements are solid state devices.

7. The apparatus of claim 5 wherein the energy source conveys energy to the one or more light elements using a wireless link.

8. The apparatus of claim 5 wherein the one or more light elements further comprise:
one or more arrays of light elements.
